(19) United States
(12) Patent Application Publication
(10) Pub. No.: US 2008/0200433 A1
(43) Pub. Date: Aug. 21, 2008

(54) Molecular Chaperone Function Regulator

(76) Inventors: Tsuyoshi Suzuki, Tokyo (JP); Akihiro Fujii, Tokyo (JP); Hideo Nakamura, Tokyo (JP); Tsutomu Yoshikawa, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 11/661,413

(22) PCT Filed: Sep. 1, 2005
(86) PCT No.: PCT/JP05/16007

§ 371 Date: Dec. 11, 2007

(30) Foreign Application Priority Data
Sep. 1, 2004 (JP) 2004-254716

(51) Int. Cl. A61K 31/69; A61K 31/497; A61K 31/517; A61K 31/5377; A61P 5/00; A61P 35/00

(52) U.S. Cl. 514/64; 514/252.17; 514/266.22; 514/234.5

(57) ABSTRACT

According to the present invention, a molecular chaperone function regulator containing a quinazoline derivative represented by the following formula (I)

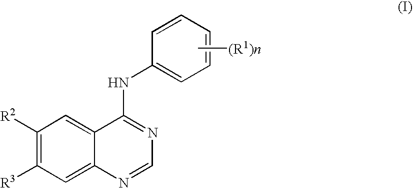

wherein each symbol is as defined in Claims, or a pharmaceutically acceptable salt thereof, a hydrate or solvate thereof, an optically active form or racemate thereof or a diastereomer mixture thereof as an active ingredient can be provided.

### TECHNICAL FIELD

The present invention relates to the prophylaxis and/or treatment of a disease involving a heat shock protein, which uses, as an active ingredient, a compound regulating the molecular chaperone function.

### BACKGROUND ART

Heat shock proteins (hereinafter sometimes indicated as HSP) are constitutively expressed in cells, and are responsible for the molecular chaperone function such as folding of and transport to the organelle of newly translated and synthesized proteins. Moreover, when cells are exposed to a stress, HSP is induced to express and prevents denaturation of proteins, or when the denaturation is remarkable, promote degradation of denatured proteins.

While examples of HSP are not limited, HSP70, HSP90 and the like can be mentioned. For example, the chaperone function of HSP90 does not operate alone but operates in conjunction with cochaperones such as HSP40, HSP70 and the like. The function is regulated by a complicated process where plural cochaperones mediate and exchange in stages during progress.

HSP90 forms a complex in a cell with various kinds of proteins and, in many cases, sustained interactions with HSP90 are essential for the stable presence and maintenance of normal functions of target proteins (hereinafter sometimes referred to as client proteins). The client proteins of HSP90 characteristically include many signaling molecules that play important roles in cell growth and cell differentiation, such as protein kinases, steroid hormone receptors and the like. Theoretically, therefore, a pharmaceutical agent that inhibits the molecular chaperone function of HSP90 also simultaneously deactivates many client proteins in the cell, and is expected to be effective for cell proliferative diseases, for example, malignant tumor.

Among the malignant tumors, particularly in breast cancer, prostate cancer and endometrial cancer that grow in a hormone-dependent manner, since both the steroid hormone receptors (receptor groups of estrogen, progesterone, androgen and the like) and cell growth-related kinases (ErbB2, Raf, Akt, Cdc2, Cdk4 etc.), included in the above-mentioned client proteins, have important functions for the survival and maintenance of the cancer cells thereof, a molecular chaperone function regulator is considered to have a high possibility of most effectively exhibiting the action. It is also considered that not only hormone-sensitive cancers but also hormone-dependent diseases such as prostatic hyperplasia, endometriosis and the like will be suitable targets of a molecular chaperone function regulator.

A determining gene responsible for chronic myelocytic leukemia, which is a representative hematopoietic tumor, is known to be a tyrosine kinase, bcr-abl, and this kinase is also a client protein of HSP90. Moreover, there is a report concluding that an HSP90 inhibitor causes apoptosis in bcr-abl positive leukemia cells and also promotes differentiation (see non-patent reference 1). Therefore, hematopoietic tumor is considered to be one of suitable target diseases for a molecular chaperone regulator.

In general, induction of molecular chaperone is said to affect survival and maintenance of tumor cells. For example, a report has documented that HSP90 shows a constitutive increase in the expression amount in various tumor cells than normal cells (see non-patent reference 2). Since HSP90 expressed in tumor cell characteristically forms a functionally more highly active chaperone complex than in normal cells, HSP90 has also been reported to be a potential object in the development of an anticancer agent (see non-patent reference 3). With regard to HSP, a clinical report reveals that the disease-free survival time is significantly short and the prognosis is very poor in patients with breast cancer who highly expressed HSP70 (see non-patent reference 4). Currently, therefore, there is a demand for a highly useful therapeutic drug or preventive drug for the treatment of malignant tumor.

A cancer thermotherapy utilizes the property of cancer cells in that they are weak against heat as compared to normal cells, and warm the whole body or tumor lesion to not less than 41° C. In general, thermotherapy is not used alone but often applied in combination with radiation or anticancer agents, in an attempt to enhance the respective treatment effects. The kind of cancer for which clinical study of thermotherapy has been actively undertaken includes malignant melanoma and soft tissue sarcoma. Soft tissue sarcoma is developed from comparatively young age and its frequent site is extremities. For induction therapy for conserving surgery of diseased limb and suppression of metastasis, clinical tests using thermotherapy and anticancer agents in combination have been vigorously reviewed (see non-patent reference 5). Accordingly, soft tissue sarcoma is considered to be one of the most suitable kinds of cancer to which to apply a hyperthermic enhancer.

When thermotherapy is repeated, tumor cells may acquire thermo tolerance to attenuate the effect. Induction of HSP70, HSP110 and the like in tumor cells reportedly relates to the acquisition of thermo tolerance and stress resistance (see non-patent reference 6). Accordingly, a pharmaceutical agent that regulates the chaperone function is expected to remove thermo tolerance of tumor cells and enhance the hyperthermic effect, thereby efficiently inhibiting the growth of tumor cells. However, almost no report exists on the study relating to the effect of hyperthermic enhancement by the chaperone regulator, and further research and development are necessary for the establishment of an effective treatment method utilizing hyperthermic enhancement.

Conventionally, benzoquinone ansamycin compounds such as herbimycin A, geldanamycin and the like are known to bind to HSP90 (see non-patent reference 7). 17-Allylamino-17-demethoxygeldanamycin (hereinafter to be indicated as 17-AAG), which is a geldanamycin derivative, is currently under clinical trials, and its effectiveness and safety have not been established.

Accordingly, a compound sold as a pharmaceutical product based on the action on HSP90 does not exist yet. Particularly, there are few compounds in the stage of clinical development of oral preparation at present.

Geldanamycin and 17-AAG are known to suppress expression of client proteins of HSP90 in tumor cells and also simultaneously increase expression of HSP70. This is considered to be the results of the conformational change from an HSP90-client complex to an HSP70-client complex prior to the proteosome degradation of client proteins (see non-patent reference 1).

On the contrary, no report has directly demonstrated regulation of molecular chaperone function by a compound having a quinazoline skeleton structure, and no finding suggestive thereof is known.

non-patent reference 1: Cancer Res. 61, 1799-804, 2001
non-patent reference 2: Int. J. Cancer 51, 613-9, 1992
non-patent reference 3: Nature 425, 407-10, 2003
non-patent reference 4: J. Natl. Cancer Inst. 85, 570-4, 1993
non-patent reference 5: Clin. Cancer Res. 5, 1650-7, 1999
non-patent reference 6: Pathol. Oncol. Res. 4, 316-21, 1998
non-patent reference 7: Cancer Cell 3, 213-7, 2003

### DISCLOSURE OF THE INVENTION

### Problems to be Solved by the Invention

In view of the current situation mentioned above, the present invention aims at providing an agent for the prophylaxis and/or treatment of a disease involving a heat shock protein.

### Means of Solving the Problems

The present inventors have conducted intensive studies in an attempt to solve the aforementioned problem and found that a certain kind of quinazoline derivative regulates the molecular chaperone function, and that the molecular chaperone function regulator is useful as a pharmaceutical composition for the prophylaxis and/or treatment of a disease involving a heat shock protein, or for a thermotherapy enhancing action, which resulted in the completion of the present invention.

Accordingly, the present invention provides the following.

The present invention relates to

(1) a molecular chaperone function regulator comprising, as an active ingredient, a quinazoline derivative represented by the following formula (I)

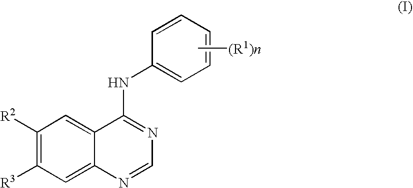

wherein n is an integer of 0 to 3, R¹ is a hydrogen atom, a halogen atom, hydroxy, cyano, nitro, trifluoromethyl, C₁₋₅ alkyl, C₁₋₅ alkoxy, —S(O)_{f}R¹³ (wherein f is an integer of 0 to 2, and R¹³ is C₁₋₅ alkyl), —NR¹⁴R¹⁵ (wherein R¹⁴ and R¹⁵ are each independently a hydrogen atom, C₁₋₅ alkyl, C₁₋₅ alkanoyl or C₁₋₅ alkylsulfonyl), C₂₋₅ alkenyl, C₂₋₅ alkynyl or C₁₋₅ alkanoyl, one of R² and R³ is
R²⁷SO₂NH— (wherein R²⁷ is C₁₋₅ alkyl optionally substituted by morpholino), (R²⁸SO₂)₂N— (wherein R²⁸ is C₁₋₅ alkyl optionally substituted by morpholino), C₁₋₅ alkoxy, CH₃COCH₂CONH—, CH₃SCH₂CH₂CONH—, NCCH₂CONH—,

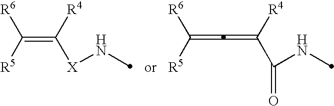

(wherein X is —C(O)— or SO₂—, and R⁴, R⁵ and R⁶ are each independently a hydrogen atom, a halogen atom or C₁₋₅ alkyl optionally substituted by a halogen atom, morpholino, 4-C₁₋₅ alkylpiperazin-1-yl or di(C₁₋₅ alkyl)amino), or

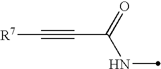

(wherein R⁷ is C₁₋₅ alkyl optionally substituted by a hydrogen atom, a halogen atom, morpholino, 4-C₁₋₅ alkylpiperazin-1-yl or di(C₁₋₅ alkyl)amino), and
the other of R² and R³ is

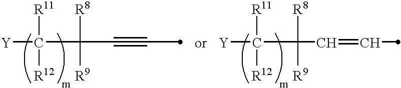

wherein R⁸ and R⁹ are each independently [a] a hydrogen atom, [b] C₁₋₅ alkyl optionally substituted by hydroxy or C₁₋₅ alkoxy, [c] R⁸ and R⁹ in combination show C═O, or [d] R⁸ and R⁹ in combination form a ring to show C₃₋₈ cycloalkylene optionally via —O—, —S— or —NR¹⁰— (wherein R¹⁰ is a hydrogen atom or C₁₋₅ alkyl), m is an integer of 0 to 3, R¹¹ and R¹² are each independently a hydrogen atom or C₁₋₅ alkyl, and
Y is a hydrogen atom, hydroxy, C₁₋₅ alkoxy, C₁₋₅ alkanoyloxy, —N(R¹⁶)—(CO)u-(CR¹⁷R¹⁸)v-(CO)j-R¹⁹ (wherein R¹⁶ is [a] a hydrogen atom or [b] C₁₋₅ alkyl optionally substituted by cyano or C₁₋₅ alkoxy, R¹⁷ and R¹⁸ are each independently a hydrogen atom or C₁₋₅ alkyl, u and j are each 0 or 1, v is an integer of 1 to 5, and R¹⁹ is a hydrogen atom, hydroxy, cyano, amino, C₁₋₅ alkoxy, morpholino, 4-C₁₋₅ alkylpiperazin-1-yl or di(C₁₋₅ alkyl)amino,

provided that when (i) u and j are simultaneously 0, then v is an integer of 2 to 5, and when (ii) R¹⁹ is a cyano group, then j is 0),

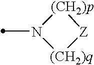

wherein p and q are each independently 2 or 3, and Z is —O—, —S(O)_{g}— (wherein g is an integer of 0 to 2), carbonyl or —NR²⁰— (wherein R²⁰ is [a] a hydrogen atom, [b] C₁₋₅ alkylsulfonyl, [c] C₁₋₅ alkanoyl, [d] C₁₋₅ alkoxycarbonyl, or [e] C₁₋₅ alkyl optionally substituted by cyano or C₁₋₅ alkoxy), or

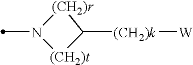

wherein r and t are each independently an integer of 1 to 3, k is 0 or 1, and W is a hydrogen atom, hydroxy, C₁₋₅ alkoxy, C₁₋₅ alkanoyloxy, carboxyl, cyano, di(C₁₋₅ alkyl)amino, morpholino, pyrrolidin-1-yl, piperidin-1-yl, 4-C₁₋₅ alkylpiperazin-1-yl or CONR²¹R²² (wherein R²¹ and R²² are each independently a hydrogen atom or C₁₋₅ alkyl), or a pharmaceutically acceptable salt thereof, or a hydrate or solvate thereof, or an optically active form or racemate thereof or a diastereomer mixture thereof,
(2) the molecular chaperone function regulator of the aforementioned (1), wherein, in the quinazoline derivative of the aforementioned formula (I),
is R² is R²⁷SO₂NH— (wherein R²⁷ is C₁₋₅ alkyl optionally substituted by morpholino), (R²⁸SO₂)₂N— (wherein R²⁸ is C₁₋₅ alkyl optionally substituted by morpholino), C₁₋₅ alkoxy, CH₃COCH₂CONH—, CH₃SCH₂CH₂CONH—, N≡CCH₂CONH—, or

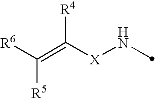

(wherein X is —C(O)— or SO₂—, and R⁴, R⁵ and R⁶ are each independently a hydrogen atom, a halogen atom or C₁₋₅ alkyl optionally substituted by a halogen atom, morpholino, 4-C₁₋₅ alkylpiperazin-1-yl or di(C₁₋₅ alkyl)amino), and

### R³ is

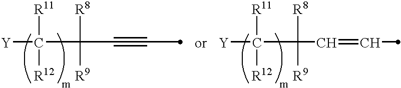

wherein m is an integer of 0 to 3,
R⁸ and R⁹ are each independently a hydrogen atom, hydroxy or C₁₋₅ alkyl optionally substituted by C₁₋₅ alkoxy, R⁸ and R⁹ in combination show —C(O)—, or R⁸ and R⁹ in combination form a ring to show C₃₋₈ cycloalkylene optionally via —O—, —S— or —NR¹⁰— (wherein R¹⁰ is a hydrogen atom or C₁₋₅ alkyl), R¹¹ and R¹² are each independently a hydrogen atom or C₁₋₅ alkyl, and
Y is a hydrogen atom, hydroxy, C₁₋₅ alkoxy, C₁₋₅ alkanoyloxy, —N(R¹⁶)—(CO)u-(CR¹⁷R¹⁸)v-(CO)j-R¹⁹ (wherein R¹⁶ is a hydrogen atom, or C₁₋₅ alkyl optionally substituted by cyano or C₁₋₅ alkoxy, R¹⁷ and R¹⁸ are each independently a hydrogen atom or C₁₋₅ alkyl, u and j are each independently 0 or 1, v is an integer of 1 to 5, and
R¹⁹ is a hydrogen atom, hydroxy, cyano, amino, C₁₋₅ alkoxy, morpholino, 4-C₁₋₅ alkylpiperazin-1-yl, or di(C₁₋₅ alkyl)amino,

provided that when (i) u and j are simultaneously 0, then v is an integer of 2 to 5, and when (ii) R¹⁹ is cyano, then j is 0),

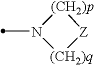

wherein p and q are each independently 2 or 3, and Z is —O—, —S(O)_{g}— (wherein g is an integer of 0 to 2), —C(O)— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or

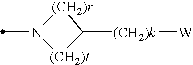

wherein r and t are each independently an integer of 1 to 3, k is 0 or 1, and W is a hydrogen atom, hydroxy, C₁₋₅ alkoxy, C₁₋₅ alkanoyloxy, carboxyl, cyano, di(C₁₋₅ alkyl)amino, morpholino, pyrrolidin-1-yl, piperidin-1-yl, 4-C₁₋₅ alkylpiperazin-1-yl or CONR²¹R²² (wherein R²¹ and R²² are each independently a hydrogen atom or C₁₋₅ alkyl),
(3) the molecular chaperone function regulator of the aforementioned (1) or (2), wherein, in the quinazoline derivative of the aforementioned formula (I),

### R³ is

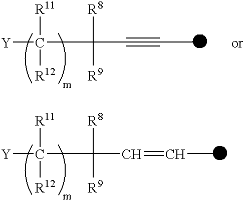

wherein m is an integer of 0 to 3,
R⁸ and R⁹ are each independently a hydrogen atom or C₁₋₅ alkyl optionally substituted by C₁₋₅ alkoxy, or R⁸ and R⁹ in combination form a ring to show C₃₋₈ cycloalkylene optionally via —O— or —NH—,
R¹¹ and R¹² are each independently a hydrogen atom or C₁₋₅ alkyl, and
Y is a hydrogen atom, hydroxy, C₁₋₅ alkoxy, C₁₋₅ alkanoyloxy, —N(R¹⁶)—(CO)u-(CR¹⁷R¹⁸)v-(CO)j-R¹⁹ (wherein R¹⁶ is a hydrogen atom, or C₁₋₅ alkyl optionally substituted by cyano or C₁₋₅ alkoxy, R¹⁷ and R¹⁸ are each independently a hydrogen atom or C₁₋₅ alkyl, u and j are each independently 0 or 1, v is an integer of 1 to 5, and R¹⁹ is a hydrogen atom, hydroxy, cyano, amino, C₁₋₅ alkoxy, morpholino, 4-C₁₋₅ alkylpiperazin-1-yl, or di(C₁₋₅ alkyl)amino, provided that when (i) u and j are simultaneously 0, then v is an integer of 2 to 5, and when (ii) R¹⁹ is cyano, then j is 0),

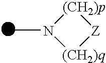

wherein p and q are each independently 2 or 3, and Z is —O—, —C(O)— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or

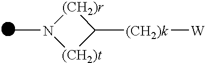

wherein r and t are each independently an integer of 1 to 3, k is 0 or 1, and W is a hydrogen atom, hydroxy, C₁₋₅ alkoxy, C₁₋₅ alkanoyloxy, carboxyl, cyano, di(C₁₋₅ alkyl)amino, morpholino or CONR²²R²² (wherein R²¹ and R²² are each independently a hydrogen atom or C₁₋₅ alkyl),
(4) the molecular chaperone function regulator of any one of the aforementioned (1) to (3), wherein, in the quinazoline derivative of the aforementioned formula (I),

### R³ is

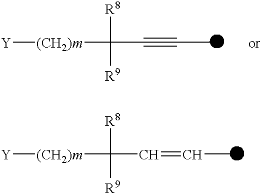

wherein m is an integer of 0 or 1,
R⁸ and R⁹ are each independently a hydrogen atom or C₁₋₅ alkyl optionally substituted by C₁₋₅ alkoxy, or R⁸ and R⁹ in combination form a ring to show C₃₋₈ cycloalkylene optionally via —O— or —NH—, and
Y is C₁₋₅ alkoxy, —N(R¹⁶)—(CO)u-(CH₂)₂—(CO)j-R¹⁹ (wherein u and j are each 0 or 1, R¹⁶ is a hydrogen atom, or C₁₋₅ alkyl optionally substituted by C₁₋₅ alkoxy, and R¹⁹ is a hydrogen atom, cyano, C₁₋₅ alkoxy, morpholino, 4-C₁₋₅ alkylpiperazin-1-yl or di(C₁₋₅ alkyl)amino, provided that when R¹⁹ is cyano, then j is 0),

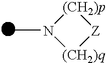

wherein p and q are each independently 2 or 3, and Z is —O— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or

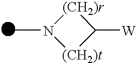

wherein r and t are each independently 1 or 2, and
W is a hydrogen atom, hydroxy, C₁₋₅ alkoxy, C₁₋₅ alkanoyloxy or carboxyl,
(5) the molecular chaperone function regulator of any one of the aforementioned (1) to (4), wherein, in the quinazoline derivative of the aforementioned formula (I),

### R³ is

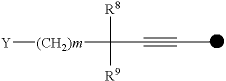

wherein m is 0 or 1,
R⁸ and R⁹ are each independently a hydrogen atom or C₁₋₅ alkyl optionally substituted by C₁₋₅ alkoxy, or R⁸ and R⁹ in combination form a ring to show C₃₋₈ cycloalkylene optionally via —O— or —NH—, and
Y is C₁₋₅ alkoxy, —N(R¹⁶)—(CH₂)₂—R¹⁹ (wherein R¹⁶ is a hydrogen atom or C₁₋₅ alkyl optionally substituted by C₁₋₅ alkoxy, and R¹⁹ is a hydrogen atom, cyano, C₁₋₅ alkoxy, morpholino, 4-C₁₋₅ alkylpiperazin-1-yl, or di(C₁₋₅ alkyl)amino, provided that when R¹⁹ is cyano, then j is 0),

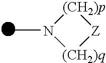

wherein p and q are each independently 2 or 3, and
Z is —O— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or

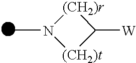

wherein r and t are each independently 1 or 2, and
W is a hydrogen atom, hydroxy, C₁₋₅ alkoxy, C₁₋₅ alkanoyloxy, carboxyl,
(6) the molecular chaperone function regulator of any one of the aforementioned (1) to (5), wherein, in the quinazoline derivative of the aforementioned formula (I),
R² is R²⁷SO₂NH— (wherein R²⁷ is C₁₋₅ alkyl), C₁₋₅ alkoxy or

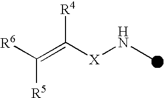

wherein each symbol is as defined above,
(7) the molecular chaperone function regulator of any one of the aforementioned (1) to (6), wherein, in the quinazoline derivative of the aforementioned formula (I), n is 1 or 2, and R¹ is a halogen atom, cyano, C₁₋₅ alkyl, C₁₋₅ alkoxy, —NR¹⁴R¹⁵ (wherein R¹⁴ and R¹⁵ are each independently a hydrogen atom, C₁₋₅ alkyl, C₁₋₅ alkanoyl or C₁₋₅ alkylsulfonyl), C₂₋₅ alkynyl, or C₁₋₅ alkanoyl,
(8) the molecular chaperone function regulator of any one of the aforementioned (1) to (7), wherein, in the quinazoline derivative of the aforementioned formula (I), n is 2, R¹ is a halogen atom, R² is

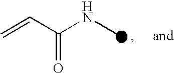

### R³ is

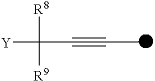

wherein R⁸ and R⁹ are each C₁₋₅ alkyl optionally substituted by C₁₋₅ alkoxy, and
Y is —N(R¹⁶)—(CH₂)₂—R¹⁹ (wherein R¹⁶ is C₁₋₅ alkyl optionally substituted by C₁₋₅ alkoxy, and R¹⁹ is di(C₁₋₅ alkyl)amino), or

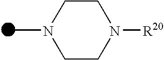

wherein R²⁰ is C₁₋₅ alkyl optionally substituted by C₁₋₅ alkoxy,
(9) the molecular chaperone function regulator of any one of the aforementioned (1) to (8), wherein the quinazoline derivative is N-{4-[(3-chloro-4-fluorophenyl)amino]-7-[3-methyl-3-(4-methyl-1-piperazinyl)-1-butynyl]-6-quinazolinyl}acrylamide bis(4-methylbenzenesulfonate,
(10) a pharmaceutical composition for the prophylaxis and/or treatment of a disease involving a heat shock protein, or for a thermotherapy enhancing action, which comprises the molecular chaperone function regulator of any one of the aforementioned (1) to (9),
(11) the pharmaceutical composition of the aforementioned (10), wherein the disease involving a heat shock protein is a hormone-dependent disease, a hematopoietic tumor or a malignant soft tissue tumor,
(12) the pharmaceutical composition of the aforementioned (11), wherein the hormone-dependent disease is prostatic hyperplasia, endometriosis or a hormone-sensitive cancer,
(13) the pharmaceutical composition of the aforementioned (12), wherein the hormone-sensitive cancer is a hormone-sensitive breast cancer, a hormone-sensitive prostate cancer or a hormone-sensitive endometrial cancer,
(14) the pharmaceutical composition of the aforementioned (11), wherein the hematopoietic tumor is leukemia, malignant lymphoma or myeloma,
(15) the pharmaceutical composition of the aforementioned (11), wherein the malignant soft tissue tumor is osteosarcoma or soft tissue sarcoma,
(16) a pharmaceutical composition for the prophylaxis and/or treatment of a disease involving a heat shock protein, or for a thermotherapy enhancing action, which comprises a quinazoline derivative represented by the following formula (I)

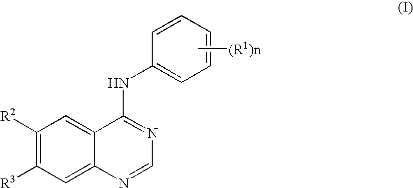

wherein each symbol is as defined in the aforementioned (1), or a pharmaceutically acceptable salt thereof, a hydrate or solvate thereof, an optically active form or racemate thereof or a diastereomer mixture thereof,
(17) the pharmaceutical composition of the aforementioned (16), wherein the disease involving a heat shock protein is a hormone-dependent disease, a hematopoietic tumor, or a malignant soft tissue tumor,
(18) the pharmaceutical composition of the aforementioned (17), wherein the hormone-dependent disease is prostatic hyperplasia, endometriosis or a hormone-sensitive cancer,
(19) the pharmaceutical composition of the aforementioned (18), wherein the hormone-sensitive cancer is a hormone-sensitive breast cancer, a hormone-sensitive prostate cancer or a hormone-sensitive endometrial cancer,
(20) the pharmaceutical composition of the aforementioned (17), wherein the hematopoietic tumor is leukemia, malignant lymphoma or myeloma, and
(21) the pharmaceutical composition of the aforementioned (17), wherein the malignant soft tissue tumor is osteosarcoma or soft tissue sarcoma.

### EFFECT OF THE INVENTION

The present invention can provide a molecular chaperone function regulator comprising, as an active ingredient, a quinazoline derivative represented by the aforementioned formula (I) or a pharmaceutically acceptable salt thereof, or a hydrate or solvate thereof, or an optically active form or racemate thereof or a diastereomer mixture thereof.

### BRIEF DESCRIPTION OF THE DRAWINGS

FIG. 1 is a graph showing the time-course changes of HSP70 induction in human epithelial cancer-derived A-431 cell, wherein the vertical axis shows the expression amount of HSP70 in the cell treated with a compound to the expression amount of HSP70 in the cell in the control well as 100, and the horizontal axis shows treatment time.

FIG. 2 shows expression of HSP70 in various tumor cells as assayed by the Western blot, wherein each lane shows C: DMSO, A: compound A, and ZD: gefinitib.

FIG. 3 shows expression of client proteins in human breast cancer-derived MCF-7 cells as assayed by the Western blot, wherein each lane shows C: DMSO, GA: geldanamycin, A: compound A, and ZD: gefinitib, as for ERα and AR, nuclear fractions were treated with respective antibodies, and as for HER2, AKT and CDK4, cell extracts were treated with respective antibodies.

FIG. 4 shows an effect of a compound on cell growth upon stimulation of human breast cancer-derived MCF-7 cells with estradiol, wherein the vertical axis shows suppression rate, the horizontal axis shows concentrations of the compound, A is compound A and TAM is tamoxifen.

FIG. 5 shows an effect of hyperthermic treatment and compound A on the growth of human prostate cancer cell-derived DU 145 cells, wherein the vertical axis shows absorbance of a compound-treated well or a heat-treated well to the absorbance of a control well of a plate free of the heat treatment as 100, and the horizontal axis shows concentrations of compound A.

FIG. 6 shows an effect of compound A on the growth of human breast cancer MCF-7 implanted into nude mouse, wherein the vertical axis shows tumor weights and the horizontal axis shows doses of compound A. 5 per group, *: p<0.05, by Dunnett's test with a solvent administration group as a control.

FIG. 7 shows an effect of compound A on ubiquitination of protein in human breast cancer-derived T-47D cell, wherein the vertical axis shows the rate of ubiquitinated protein treated with a compound to the control as 100 and the horizontal axis shows treatment time.

### BEST MODE FOR EMBODYING THE INVENTION

Respective definitions in the present invention are as follows.

The compound of the present invention is a quinazoline derivative represented by the aforementioned formula (I).

As the halogen atom defined for each substituent of the aforementioned formula (I), fluorine atom, chlorine atom, bromine atom and iodine atom can be mentioned; as the C₁-C₅ alkyl group, methyl group, ethyl group, n-propyl group, iso-propyl group, n-butyl group, iso-butyl group, sec-butyl group, tert-butyl group, n-pentyl group, neopentyl group and the like can be mentioned; as the C₁-C₅ alkoxy group, methoxy group, ethoxy group, n-propoxy group, iso-propoxy group, n-butoxy group, iso-butoxy group, sec-butoxy group, tert-butoxy group, n-pentyloxy group, neopentyloxy group and the like can be mentioned; as the C₂-C₅ alkenyl group, vinyl group, 1-propenyl group, 2-propenyl group, 1-butenyl group, 2-methylpropen-1-yl group, 2-butenyl group, 1-pentenyl group, 2-pentenyl group and the like can be mentioned; as the C₂-C₅ alkynyl group, ethynyl group, 1-propynyl group, 1-butynyl group, 1-pentynyl group and the like can be mentioned; as the C₁-C₅ alkanoyl group, formyl group, acetyl group, propionyl group, butyryl group, isovaleryl group, valeryl group and the like can be mentioned; as the C₁-C₅ alkylsulfonyl group, methylsulfonyl group, ethylsulfonyl group and the like can be mentioned; as the C₃-C₅ cycloalkylene group, cyclopropane group, cyclobutane group, cyclopentane group, cyclohexane group and the like can be mentioned, as the C₁-C₅ alkanoyloxy group, acetyloxy group, propionyloxy group and the like can be mentioned, as the C₁-C₅ alkylsulfonyl group, methylsulfonyl group, ethylsulfonyl group and the like can be mentioned, and as the C₁-C₅ alkoxycarbonyl group, methoxycarbonyl group, ethoxycarbonyl group and the like can be mentioned.

The quinazoline derivative of the present invention is converted to a salt with the corresponding acid or base by a known method.

Examples of the salt include inorganic acid salts such as hydrochloride, sulfate, carbonate, phosphate and the like, and organic acid salts such as formate, acetate, propionate, lactate, oxalate, fumarate, maleate, citrate, tartrate, benzoate, phthalate, methanesulfonate, p-toluenesulfonate, isethionate, glucuronate, gluconate and the like. In addition, alkali metal salts such as sodium salt, potassium salt and the like, alkaline earth metal salts such as magnesium salt, calcium salt and the like, ammonium salt, a salt with a pharmacologically acceptable organic amine (tetramethylamine, triethylamine, benzylamine, phenethylamine, monoethanolamine, diethanolamine, tris(hydroxyethylamine), lysine and arginine etc.) can be mentioned.

The quinazoline derivative of the present invention can have various steric structures. For example, when considered from an asymmetric carbon atom as a center, the absolute configuration thereof may be (S)-form or (R)-form, or a racemate. Pure forms of optical isomer and diastereoisomer, optional mixtures of the isomers, racemate and the like are all encompassed in the present invention.

The quinazoline derivative of the formula (I) can be present in the form of, for example, a solvate such as hydrate or a non-solvate, and the present invention encompasses all such kinds of solvates having an anticancer activity.

Preferable embodiments of the compounds of the present invention are shown in the following Tables 1-9. In the Tables, Me means methyl group, Et means ethyl group and Pr means propyl group.

TABLE 1

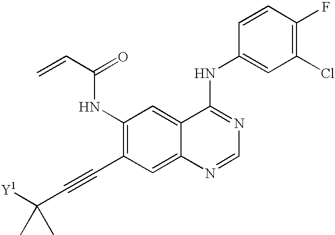

Y¹

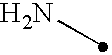

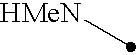

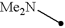

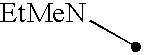

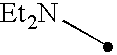

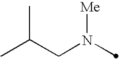

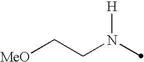

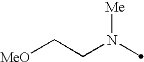

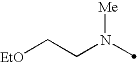

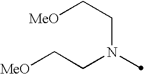

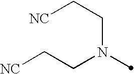

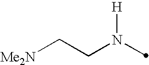

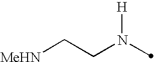

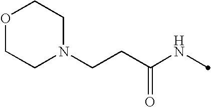

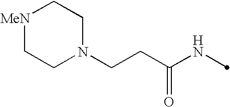

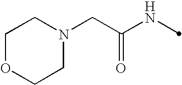

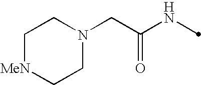

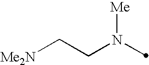

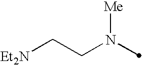

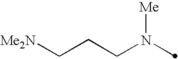

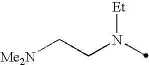

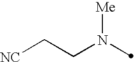

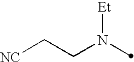

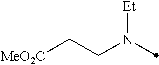

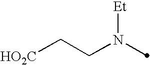

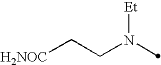

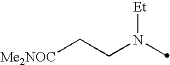

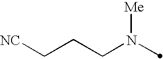

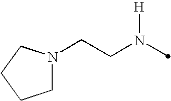

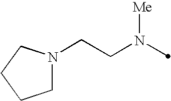

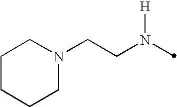

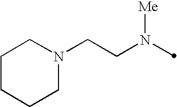

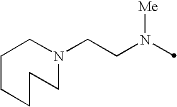

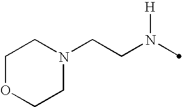

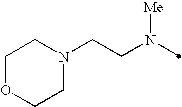

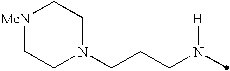

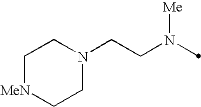

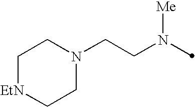

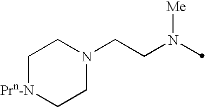

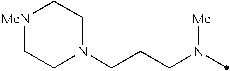

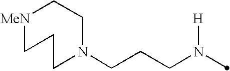

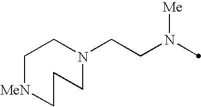

TABLE 2

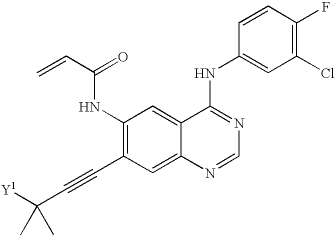

Y¹

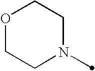

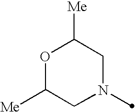

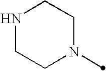

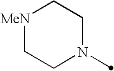

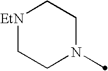

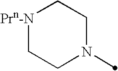

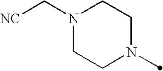

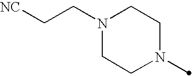

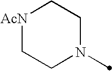

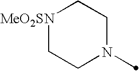

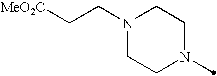

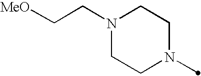

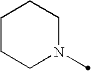

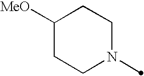

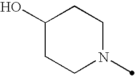

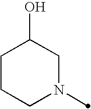

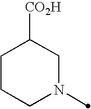

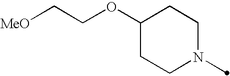

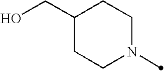

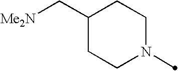

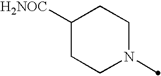

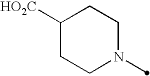

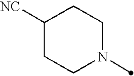

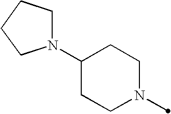

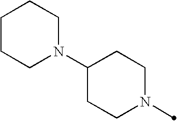

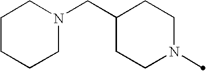

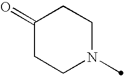

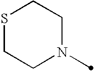

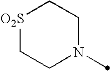

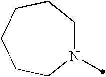

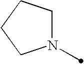

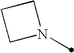

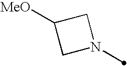

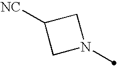

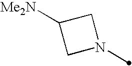

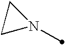

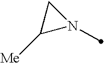

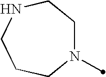

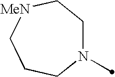

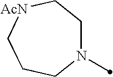

TABLE 3

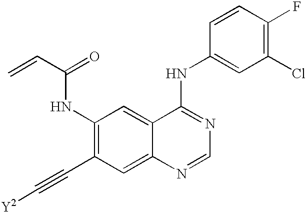

Y²

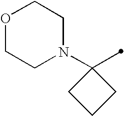

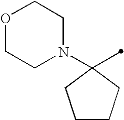

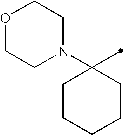

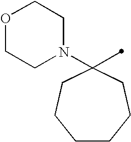

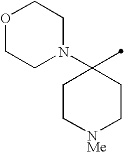

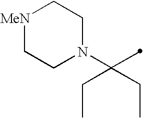

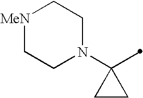

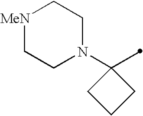

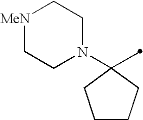

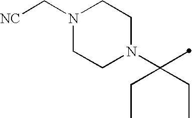

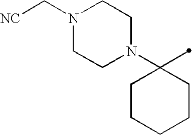

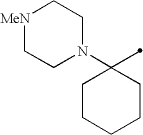

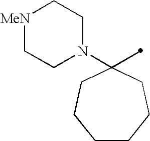

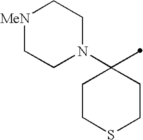

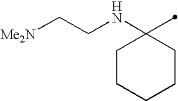

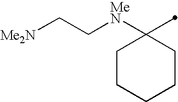

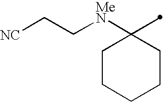

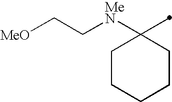

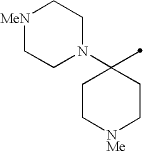

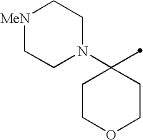

TABLE 4

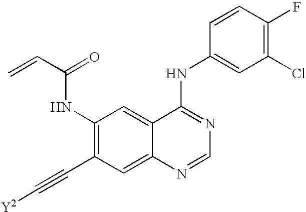

Y²

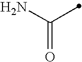

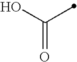

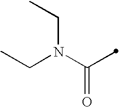

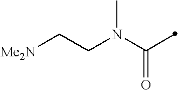

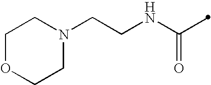

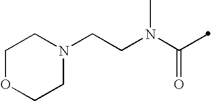

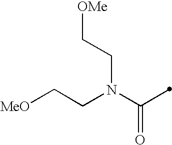

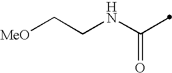

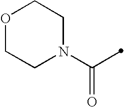

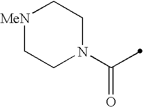

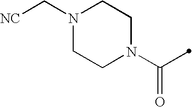

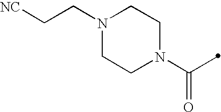

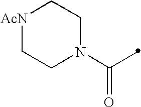

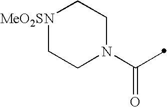

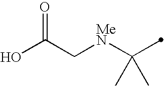

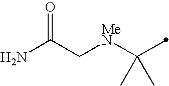

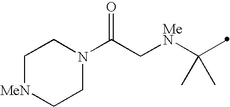

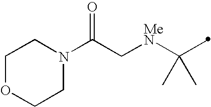

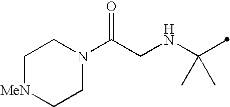

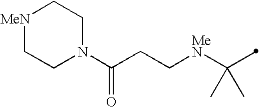

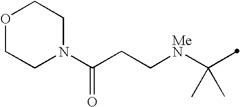

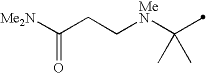

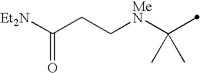

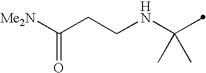

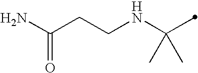

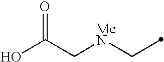

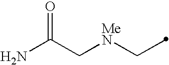

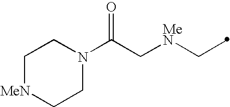

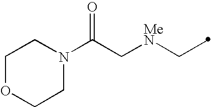

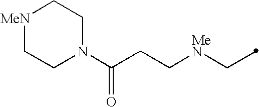

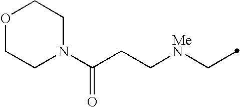

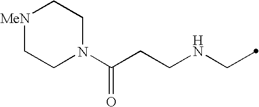

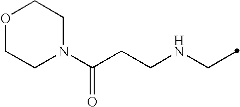

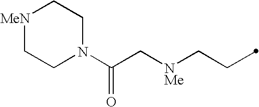

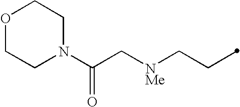

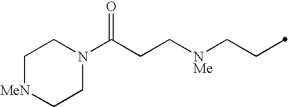

TABLE 5

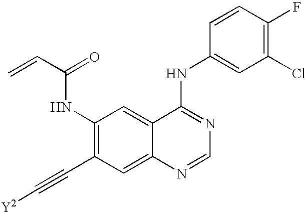

Y²

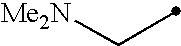

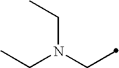

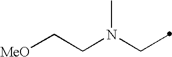

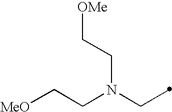

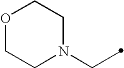

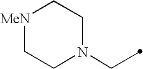

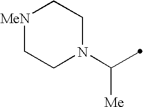

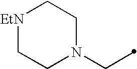

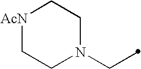

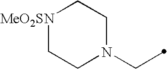

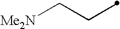

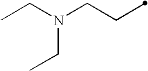

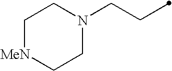

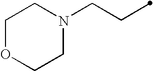

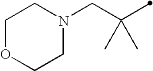

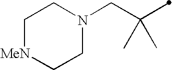

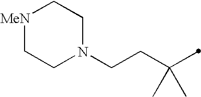

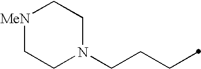

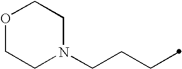

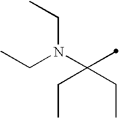

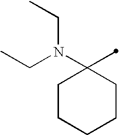

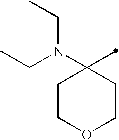

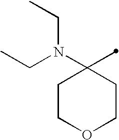

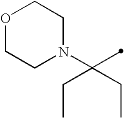

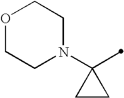

TABLE 6

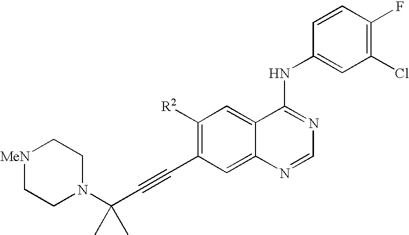

R²

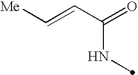

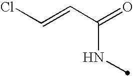

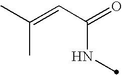

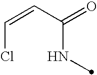

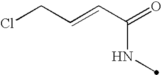

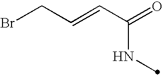

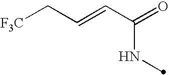

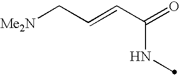

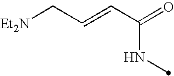

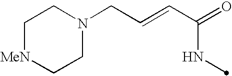

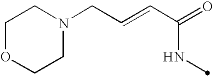

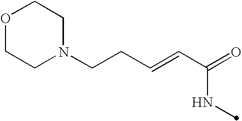

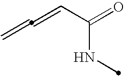

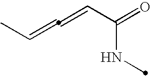

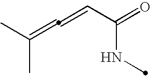

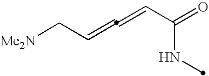

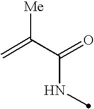

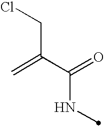

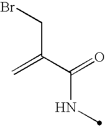

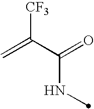

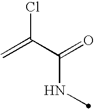

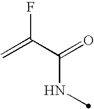

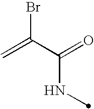

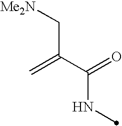

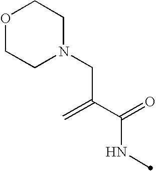

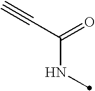

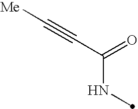

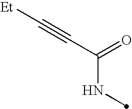

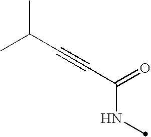

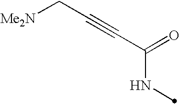

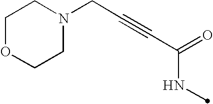

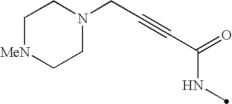

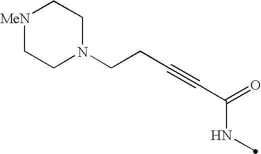

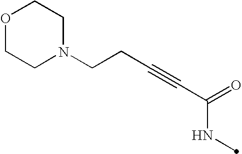

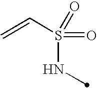

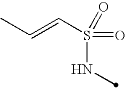

TABLE 7

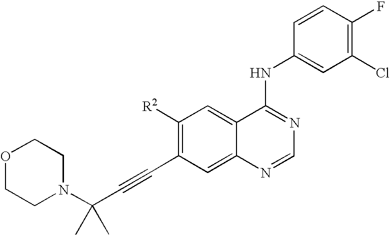

R²

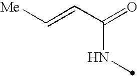

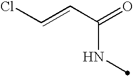

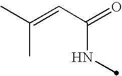

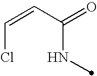

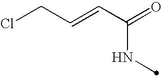

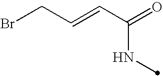

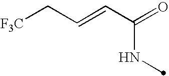

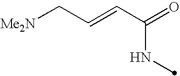

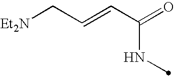

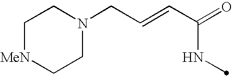

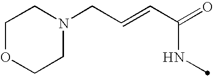

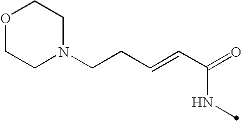

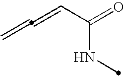

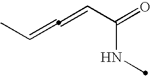

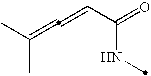

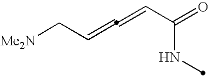

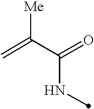

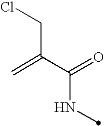

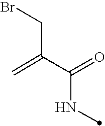

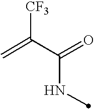

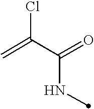

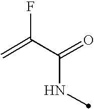

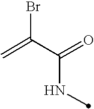

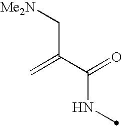

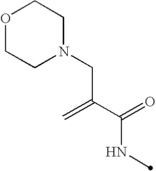

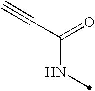

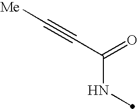

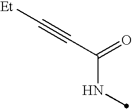

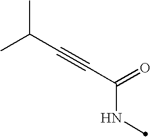

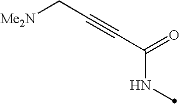

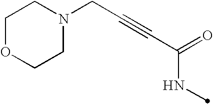

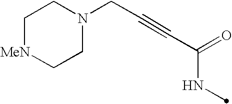

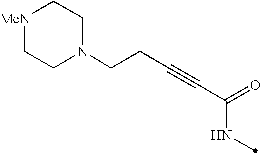

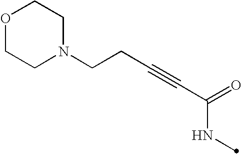

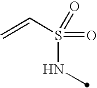

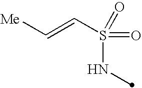

TABLE 8

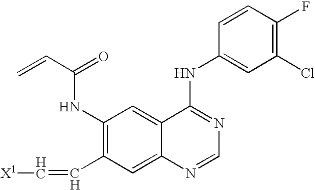

X¹

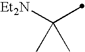

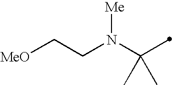

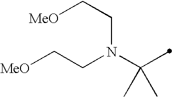

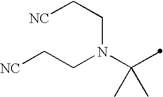

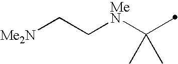

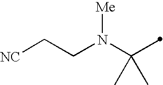

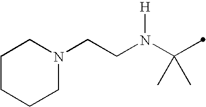

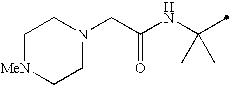

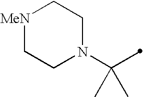

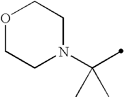

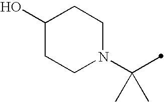

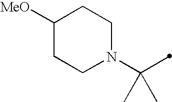

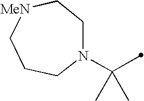

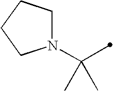

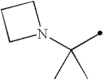

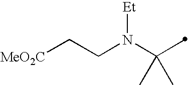

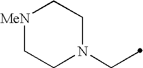

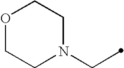

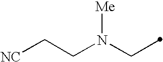

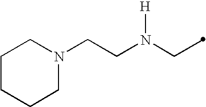

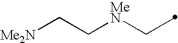

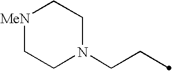

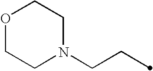

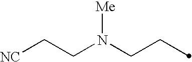

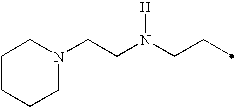

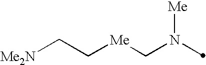

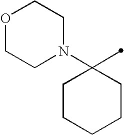

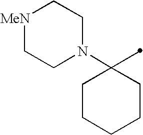

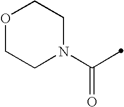

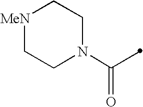

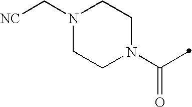

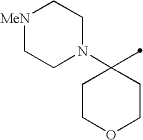

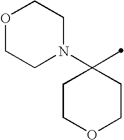

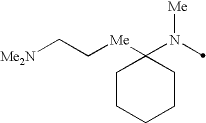

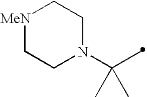

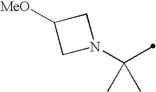

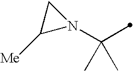

TABLE 9

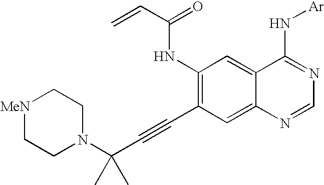

Ar

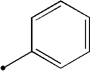

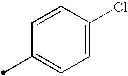

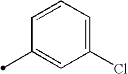

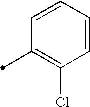

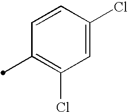

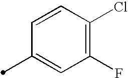

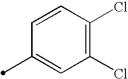

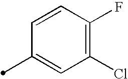

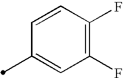

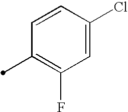

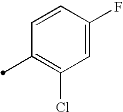

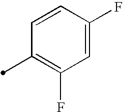

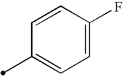

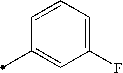

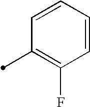

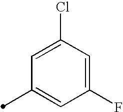

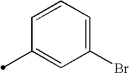

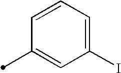

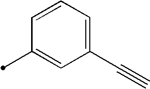

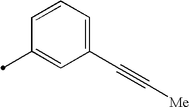

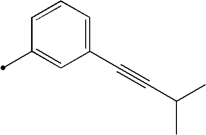

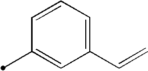

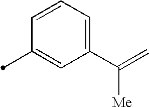

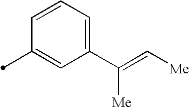

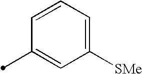

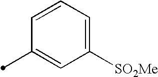

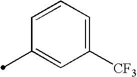

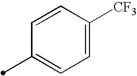

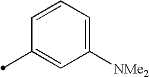

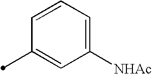

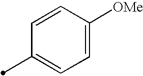

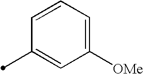

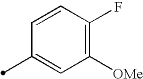

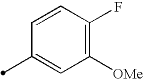

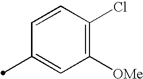

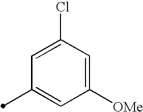

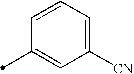

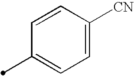

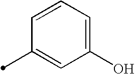

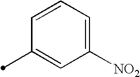

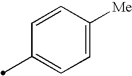

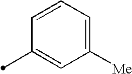

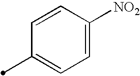

In the present invention, the “heat shock protein” may be any suitable heat shock protein (HSP) or a complex thereof, and is preferably HSP70 and HSP90.

In the present invention, the “regulation of molecular chaperone function” means promotion of inhibition of the molecular chaperone function and degradation of client proteins through regulation of expression amount of HSP itself or action on the formation of a HSP complex including client proteins.

In the present invention, specific examples of the “disease involving a heat shock protein” include hormone-dependent diseases, hematopoietic tumors and malignant soft tissue tumors.

In the present invention, the “hormone-dependent disease” is a disease requiring hormone for the pathology activity of the disease, and is a suitable disease wherein steroid hormone is involved in the pathology activity. Specifically, prostatic hyperplasia, endometriosis, hormone-sensitive cancer and the like can be mentioned.

In the present invention, specific examples of the “hormone-sensitive cancer” include hormone-sensitive breast cancer, hormone-sensitive prostate cancer and hormone-sensitive endometrial cancer.

Examples of the aforementioned compound represented by the formula (I) in the present specification include quinazoline compounds described in WO02/66445, and the production method of the compounds is described in the above-mentioned specification.

Moreover, a pharmaceutically acceptable salt of the aforementioned formula (I), a hydrate thereof, a solvate thereof, an optically active form or racemate thereof and a diastereomer mixture thereof can be synthesized by a conventional method.

The aforementioned compound of the formula (I) obtained by the above-mentioned method, a pharmaceutically acceptable salt thereof, a hydrate or solvate thereof, an optically active form or racemate thereof and a diastereomer mixture thereof are useful as molecular chaperone function regulators, useful as drugs for the prophylaxis and/or treatment of a disease involving a heat shock protein, and further useful as thermotherapy enhancing drugs.

While one or more kinds of the aforementioned compound of the formula (I) or a salt thereof, which is the active ingredient, may be directly administered to patients as the prophylactic and/or therapeutic agent of the present invention, preferably, a pharmacologically and pharmaceutically acceptable additive for the active ingredient is added, and the mixture should be provided in the form of a preparation well known to those of ordinary skill in the art.

As the pharmacologically and pharmaceutically acceptable additive, for example, excipient, disintegrant or disintegration aids, binder, lubricant, coating agent, dye, diluent, base, dissolving agent and dissolution aids, isotonicity agent, pH regulator, stabilizer, spray, adhesive and the like can be used. Examples of the preparation suitable for oral administration include tablet, capsule, powder, fine granule, granule, liquid, syrup and the like and examples of the preparation suitable for parenteral administration include injection, drip infusion, suppository and the like.

The preparation suitable for oral administration can contain excipient, disintegrant, disintegration aids, binder, lubricant, coating agent, base and the like as additives.

The preparation suitable for injection or infusion can contain additives for preparation, such as dissolving agent, dissolution aids, isotonicity agent, pH regulator and the like.

Moreover, the molecular chaperone function regulator of the present invention can be used in combination with an anticancer agent such as 5-fluorouracil, gemcitabine, doxorubicin, irinotecan, cisplatin, paclitaxel, vincristine, etoposide, trastuzumab, imatinib and the like, a hormonal therapeutic agent such as leuprorelin, tamoxifen, anastrozole, goserelin and the like, an antiemetic agent such as granisetron, dexamethasone, metoclopramide and the like, and the like.

The administration route of the pharmaceutical agent of the present invention is not particularly limited, and the agent can be orally or parenterally administered. For the disease involving HSP, for example, intravenous administration, intraarterial administration or intracardiac injection for the purpose of preventing aggravation of the condition, ameliorating the symptoms and the like is preferable.

While the dose of the pharmaceutical agent of the present invention can be appropriately determined according to the desired prophylaxis and/or treatment of the disease involving heat shock proteins, conditions such as age and state of patients and the like, generally, about 0.001-100 mg/kg is preferably administered to an adult by injection or infusion once a day or in two or more divided portions, or about 0.001-100 mg/kg is preferably administered orally to an adult once a day or in two or more divided portions.

### EXAMPLES

The present invention is explained in detail in the following by referring to Examples, which are not to be construed as limitative as long as they do not go beyond the gist of the invention.

The reagents, compounds and tumor cells used for the experiment of each Example were as follows. HSP70 specific antibody: manufactured by StressGen Biotechnologies Corp., #SPA-810

anti-HER2 antibody: manufactured by NeoMarkers Inc., Ab-15
anti-EGFR antibody: manufactured by Upstate Inc., #06-129
anti-ERα antibody: manufactured by Santa Cruz Biotechnology, Inc., sc-7207
anti-AR antibody: manufactured by Santa Cruz Biotechnology, Inc., sc-815
anti-AKT antibody: manufactured by Cell Signaling Technology, Inc., #9272
anti-CDK4 antibody: manufactured by Upstate Inc., #06-139
anti-ubiquitin antibody: manufactured by Santa Cruz Biotechnology, Inc., sc-8017
compound A: The following compounds were synthesized according to the description of WO02/66445 and used for the experiment.

### N-{4-[(3-Chloro-4-fluorophenyl)amino]-7-[3-methyl-3-(4-methyl-1-piperazinyl)-1-buthynyl]-6-quinazolinyl}acrylamide bis(4-methylbenzenesulfonate)}

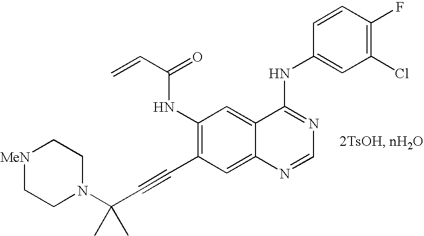

Compounds B-F: The following compounds were synthesized according to the description of WO02/66445 and used for the experiment.

Compound B:

### N-([4-(3-Chloro-4-fluorophenyl)amino]-7-{3-[4-(2-methoxyethyl)-1-piperazinyl]-3-methyl-1-butynyl}-6-quinazolinyl)acrylamide

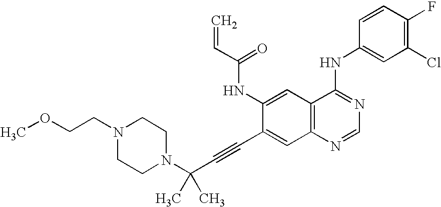

Compound C:

### N-{4-[(3-Chloro-4-fluorophenyl)amino]-7-[3-(4-methyl-1-piperazinyl)-1-propynyl]-6-quinazolinyl}acrylamide

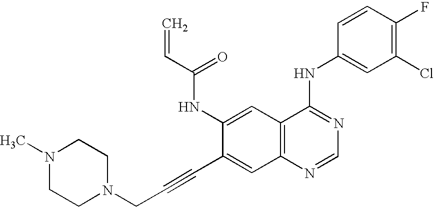

Compound D:

### N-{4-[(3-Cyano-phenyl)amino]-7-[3-methyl-3-(4-methyl-1-piperazinyl)-1-butynyl]-6-quinazolinyl}acrylamide

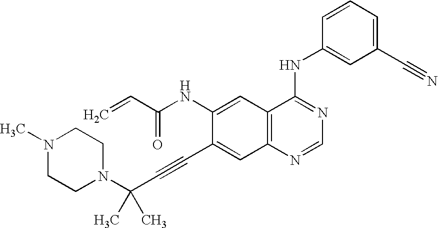

Compound E:

### N-{4-[(3-Chloro-4-fluorophenyl)amino]-7-[4-(4-methyl-1-piperazinyl)-4-tetrahydropyranylethynyl]-6-quinazolinyl}acrylamide

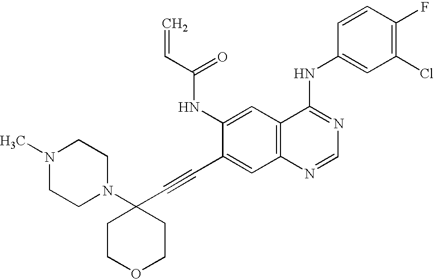

Compound F:

### (3-Chloro-4-fluorophenyl)-(7-ethoxy-6-{3-methyl-3-[methyl-(1-methyl-4-piperidinyl)amino]-1-butynyl}-4-quinazolinyl)amine

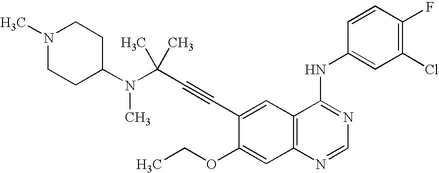

gefinitib: The following compound having a quinazoline skeleton and known as an anti-malignant tumor agent was synthesized according to the description of WO96/33980 and used for the experiment as a control drug.

### 4-(3′-Chloro-4′-fluoroanilino)-7-methoxy-6-(3-morpholinopropoxy)quinazoline

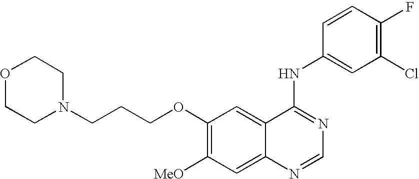

geldanamycin: Cat. #G 3381, obtained from Sigma Ltd.

bortezomib: The following compound known as a proteasome inhibitor was synthesized according to the description of U.S. Pat. No. 6,083,903 and used for the experiment as a control drug.

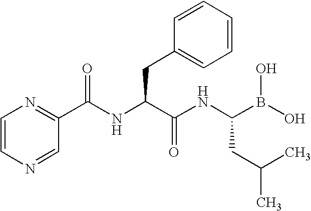

XTT (2,3-bis(2-methoxy-4-nitro-5-sulfophenyl)-2H-tetrazolium-5-carboxanilide) was used as a reagent: Cat. #X 4251, obtained from Sigma Ltd.
A-431 cell: human epithelial cancer-derived A-431 cell, obtained from Cell Resource Center for Biomedical Research Institute of Development, Aging and Cancer, Tohoku University School of Medicine (hereinafter indicated as Cell Resource Center).
TE-8 cell: human esophagus cancer-derived TE-8 cell (obtained from Cell Resource Center)
NCI-H520 cell: human lung cancer-derived NCI-H520 cell (obtained from ATCC)
HPAC cell: human pancreas cancer-derived HPAC cell (obtained from ATCC)
DU145 cell: human prostate cancer-derived DU145 cell (obtained from ATCC)
KPL-4 cell: human breast cancer-derived KPL-4 cell (provided by Mr. KUREBAYASHI Junichi, Kawasaki Medical School, reference: Kurebayashi, J., et al., British J Cancer, 79, 707-717, 1999)
MKN-45 cell: human stomach cancer-derived MKN-45 cell (obtained from Health Science Research Resources Band)
DLD-1 cell: human colon cancer-derived DLD-1 cell (obtained from ATCC)
MCF-7 cell: human breast cancer-derived MCF-7 cell (obtained from ATCC)
T-47D cell: human breast cancer-derived T-47D cell (obtained from ATCC)

### Example 1

### Time-Dependent HSP70 Induction Using Human Epithelial Cancer-Derived A-431 Cell

(Method) A-431 cells were cultured in a 12-well plate and, at the time point when the cells grew to about 70-80%, compound A and a control drug gefinitib were each added to 10 μM.

DMSO (0.1%) was added to the control. After the addition, the cells were collected 6, 9, 12 and 24 hr later and cell extracts were prepared. Expression of HSP70 protein in the cell extracts was examined by Western blot using an HSP70 specific antibody. T/C (%) was determined using the amount of chemical luminescence on a PVDF film due to an HRP-labeled secondary antibody as the HSP70 expression amount and HSP70 expression amount of the cells in the control well as 100.

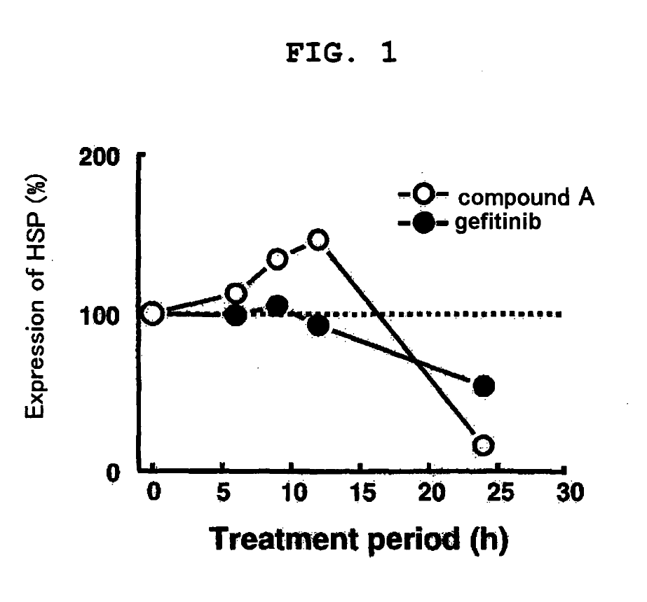

The results are shown in FIG. 1.

(Results) In A-431 cells treated with compound A (—O—), the expression of HSP70 increased in a time-dependent manner during the period of from 6 hr to 12 hr after the treatment, and markedly decreased 24 hr after the treatment. The decrease was assumed to have been caused by the cytotoxic effect of compound A. On the other hand, in A-431 cells treated with gefinitib (-•-), the expression of HSP70 did not show a clear increase until after 12 hr, and the expression of HSP70 decreased after 24 hr from the treatment.

From the results, it has been clarified that compound A induces HSP70. Moreover, it has been clarified that HSP70 is induced after 6 hr from the treatment with compound A.

### Example 2

### Induction of HSP70 in Various Human Tumor-Derived Cells

(Method) As to the induction of HSP70 by compound A, induction in various human tumor cells was examined.

TE-8 cell, NCI-H520 cell, HPAC cell, DU145 cell, KPL-4 cell, MKN-45 cell, HL-60 cell and DLD-1 cell were each were cultured in a 12-well plate.

At the time point when the cells grew to about 70-80%, compound A and a control drug gefinitib were each added to 10 μM. DMSO (0.1%) was added to a control well. After the addition, the cells were collected 9 hr later and cell extracts were prepared. Expression of HSP70 protein in the tumor cell extracts was examined by Western blot using an HSP70 specific antibody.

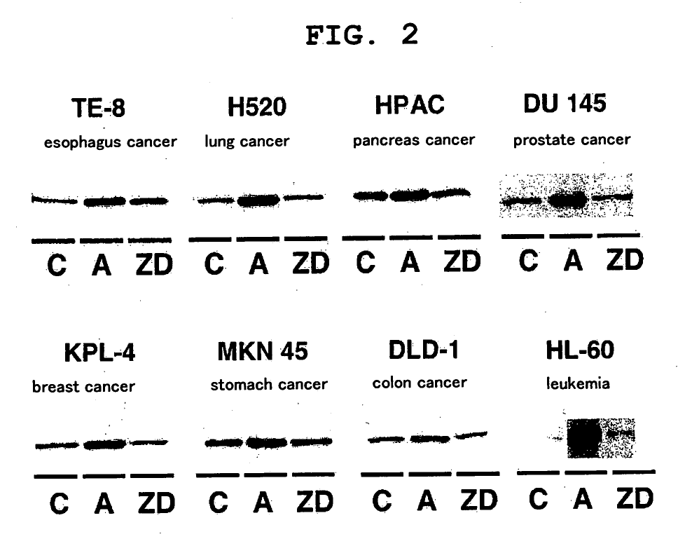

The results are shown in FIG. 2.

(Results) HSP70 expression at 9 hr after compound A treatment increased in any human tumor-derived cells as compared to the DMSO-treated control. In contrast, the gefinitib-treated tumor cells did not show a clear increase in the HSP70 expression.

From the results, it has been clarified that compound A induces HSP70 in a broad range of tumor cells.

### Example 3

### Suppressive Action on HSP90 Client Protein Expression

(Method) Effect of compound A on the expression of client proteins EGFR, HER2, ERα, AR, AKT and CDK4 of HSP90 chaperone was examined.

MCF-7 cells were cultured in a culture dish (diameter 10 cm) and, at the time point when the cells grew to about 70-80%, compound A and a control drug gefinitib were each added to 10 μM. Geldanamycin (1 μM) was added to the positive control and DMSO (0.1%) was added to the negative control.

After the addition of the compound, the cells were collected and divided in two 24 hr later. Cell extracts were prepared from half the cells, and cytoplasmic fraction and nuclear fraction were prepared from the remaining half. The cytoplasmic fraction and nuclear fraction were prepared using NER-PER (trademark) Nuclear and Cytoplasmic Extraction Reagents (manufactured by PIERCE, #78833). Expression of client in each tumor cell extract and nuclear fraction was examined by Western blot using an antibody specific to each client.

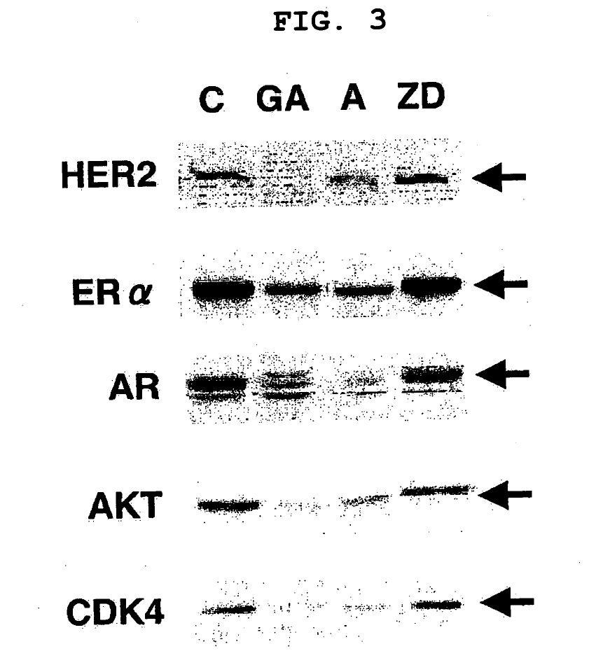

The results are shown in FIG. 3.

(Results) All clients showed decreased expression in the geldanamycin treatment group and compound A-treated cells, as compared to DMSO-treated control cells. In contrast, a clear decrease of client was not observed in the gefinitib-treated tumor cells. From the results, it has been suggested that compound A inhibits molecular chaperone function.

### Example 4

### Suppressive Effect on Estradiol (E2)-Dependent Cell Growth

(Method) Using human breast cancer-derived MCF-7 cell that expresses estrogen receptor and grows in response to estradiol (E2, 17β-Estradiol, Cat. #E 1024, Sigma Ltd.), the effect of compound A on the cell growth in the presence of E2 was examined. As the control compound, tamoxifen (Cancer Res., 62, 2474-2477, 2002) (Cat. #T 9262, Sigma Ltd.) known to suppress growth of human breast cancer-derived MCF-7 cell by E2 stimulation was used. MCF-7 cells were suspended in a phenol red non-addition DME-F12 mixed medium containing 5% fetal calf serum treated with activated carbon, and cultured at 2×10⁴ cells/well in a 24 well plate. The next day (Day 1), E2 was added to half the number of wells in the plate to the final concentration of 10 nM and the medium alone was added to the rest of the wells during medium exchange. Compound A was added to the wells at 0.037, 0.11, 0.33, 1 and 3 μM, and tamoxifen was added to the wells at 0.062, 0.19, 0.56, 1.7 and 5 μM.

On Day 4 and Day 7, the medium was discarded, a medium having the same composition was added and the cells were continuously cultured. The medium alone was added to the control wells.

The cell growth activity was measured on Day 10 by the dye method using XTT.

That is, after the completion of the culture, XTT was added to each well and, after color development for 4 hr, the reaction mixture was transferred to a 96 well plate, and the absorbance at 492 nm was measured.

The suppression rate was calculated from the following formula.

Suppression rate (%)={1−[(absorbance of compound addition well in the presence of E2)−(absorbance of control well without E2 addition)]÷[(absorbance of control well in the presence of E2)−(absorbance of control well without E2 addition)]×100

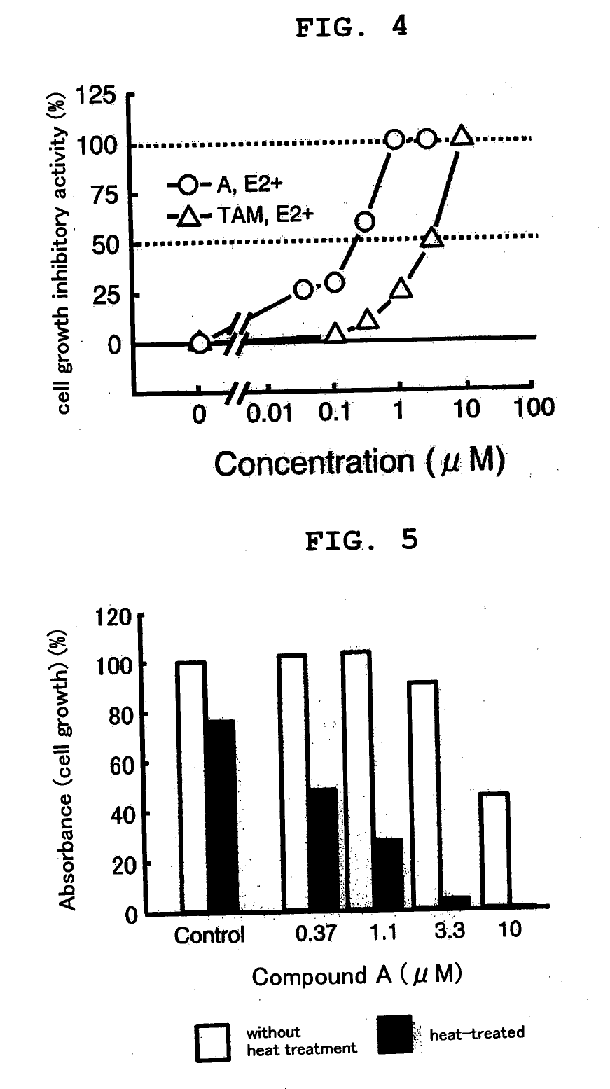

The results are shown in FIG. 4.

(Results) It has been clarified that compound A (—◯—) suppresses the growth of human breast cancer-derived MCF-7 cells by E2 stimulation, as in the case of tamoxifen (-Δ-).

### Example 5

### Cell Growth Suppression Enhancing Action by Combined Use of Hyperthermic Treatment and Compound A

(Method) Using human prostate cancer-derived DU 145 cells, the effect of compound A on the cell growth suppressive action during hyperthermic treatment was examined. DU 145 cells were cultured at 1×10⁴ cells/well in two 96 well plates. The next day, compound A was added to each plate to 0.37, 1.1, 3.3 and 10 μM, one plate was incubated for 4 hr in a 5% CO₂ incubator set to 43° C. (heat treatment), and the other plate was incubated in the same manner for 4 hr in a 5% CO₂ incubator set to 37° C. After 4 hr, the wells were washed 3 times with saline, a growth medium was added and cultured in a 5% CO₂ incubator at 37° C. for 24 hr.

The cell growth activity was measured by the dye method using XTT as in Example 4. That is, after the culture, XTT was added to each well and, 4 hr later, the absorbance at 492 nm was measured.

T/C (%) was determined from the following formula with the absorbance of the control well in the plate without the heat treatment as 100.

T/C (%)=[absorbance of heat-treated control well or compound-treated well÷absorbance of control well without heat treatment]×100

The results are shown in FIG. 5.

(Results) From the results, it has been clarified that the cell death due to heat shock is enhanced by increasing the concentration of compound A.

### Example 6

### Antitumor Effect of Compound A on Human Breast Cancer-Derived MCF-7 Implanted in Nude Mouse

(Method) Human breast cancer-derived MCF-7 cells were subcutaneously implanted in female nude mouse purchased from CLEA Japan, Inc. The effect of compound A on the growth was examined. A 17β-estradiol solution was added dropwise to a small piece of glass microfibre filter GF/B (Whatman, #1821 05) to give a pellet (500 μg/filter). The pellet was subcutaneously implanted in the neck of female Balb/c nude mouse, and MCF-7 cells were subcutaneously implanted in the right back at 5×10⁶ cells/mouse. From 7 days after the implantation, compound A was orally administered at 30 mg/kg and 100 mg/kg to nude mice (5 per group) once a day for 2 weeks.

The 0.5% tragacanth solution used as a solvent was similarly administered to the control. The next day of the final administration, the nude mice were sacrificed under anesthesia, and the subcutaneous tumor was isolated and weighed. The mean tumor weight and standard error of each group were calculated, and statistically analyzed by the Dunnett's test and using the solvent administration group as a control.

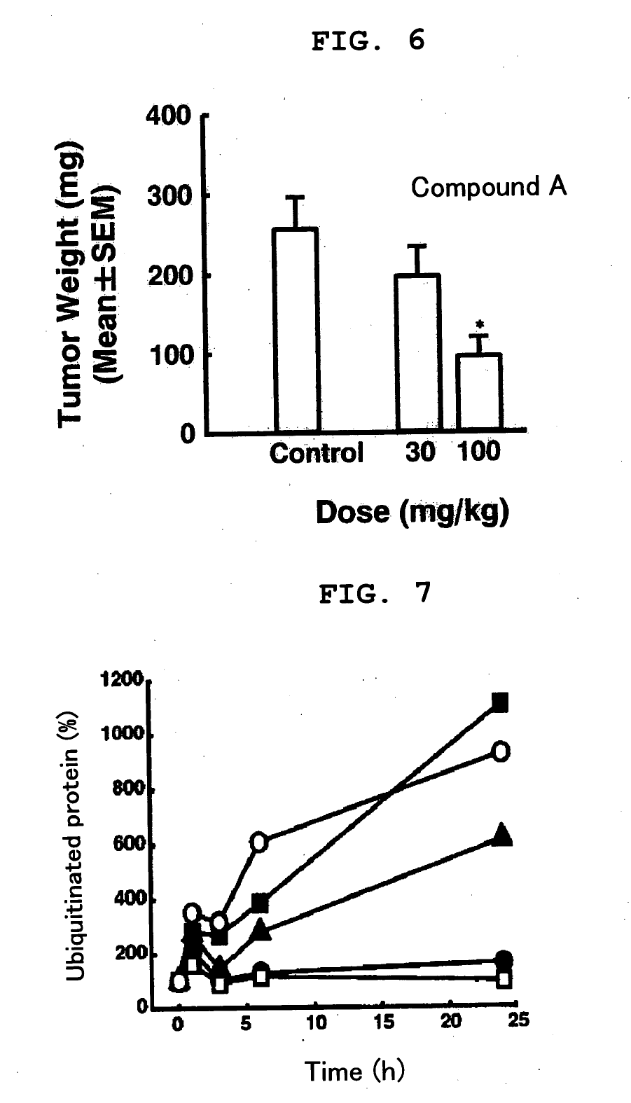

The results are shown in FIG. 6.

(Results) As shown in FIG. 6, the tumor weight significantly decreased in the group administered with 100 mg/kg of compound A as compared to a control group.

From the results, it has been clarified that compound A shows an antitumor effect on MCF-7 breast cancer grown with estrogen.

### Example 7

### Induction of HSP70 by Compounds A-F Using Human Breast Cancer-Derived T-47D Cells

(Method) T-47D cells were cultured in a 12-well plate and, at the time point when the cells grew to about 70-80%, compounds A, B, C, D, E and F were each added to 10 μM.

DMSO (0.1%) was added to the control. After the addition, the cells were collected 9 hr later and cell extracts were prepared. Expression of HSP70 protein in the cell extracts was examined by Western blot using an HSP70 specific antibody. T/C (%) was determined using the amount of chemical luminescence on a PVDF film due to an HRP-labeled secondary antibody as the HSP70 expression amount and HSP70 expression amount of the cells in the control well as 100.

The results are shown in Table 10.

TABLE 10

| | Experiment 1 | Experiment 2 |
|---|---|---|
| compound | (T/C, %) | (T/C, %) |
| control | 100 | 100 |
| A | 299 | 895 |
| B | | 482 |
| C | 207 | |
| D | | 446 |
| E | 172 | |
| F | 141 | |

(Results) Expression of HSP70 increased in T-47D cells treated with compounds A-F, as compared to the control.

From the results, it has been shown that HSP70 is induced in the cell by treating T-47D cells with compounds A-F for 9 hr.

### Example 8

### Induction of Ubiquitinated Protein by Compound A in Human Breast Cancer-Derived T-47D Cell

(Method) T-47D cells were cultured in a 6-well plate and, at the time point when the cells grew to about 70-80%, compound A was added to 1, 3 and 10 μM and bortezomib (PS-341) and geldanamycin (GA) were each added to 3 μM. DMSO (0.1%) was added to the control. After the addition, the cells were collected 1, 3, 6 and 24 hr later and cell extracts were prepared. Expression of ubiquitinated protein in the cell extracts was examined by Western blot using a ubiquitin specific antibody. T/C (%) was determined using the amount of chemical luminescence on a PVDF film due to an HRP-labeled secondary antibody as the ubiquitinated protein expression amount and the expression amount of the cell extract in the control well as 100.

The results are shown in FIG. 7.

(Results) In bortezomib (-◯-)-treated T-47D cells, a ubiquitinated protein remarkably appeared from 3 hr after the treatment. In T-47D cells treated with compound A 1 μM (-•-) and geldanamycin (-□-), ubiquitination of protein did not occur until after 24 hr. In contrast, in T-47D cells treated with compound A 3 (-▴-) μM and 10 μM (-□-), the amount of ubiquitinated protein increased from 6 hr after the treatment, which lasted until after 24 hr from the treatment.

From the results, it has been clarified that ubiquitinated protein appears from 6 hr later, by treating with compound A (3 μM and 10μ).

### INDUSTRIAL APPLICABILITY

According to the present invention, a molecular chaperone function regulator comprising a quinazoline derivative represented by the aforementioned formula (I) or a pharmaceutically acceptable salt thereof, a hydrate or solvate thereof, an optically active form or racemate thereof or a diastereomer mixture thereof as an active ingredient can be provided.

This application is filed claiming priority right based on a patent application No. 2004-254716 filed in Japan.

1. A method for the prophylaxis and/or treatment of a disease involving a heat shock protein, or for a thermotherapy enhancing action, which comprises administering to a patient in need thereof a molecular chaperone function regulator comprising, as an active ingredient, a quinazoline derivative represented by the following formula (I) 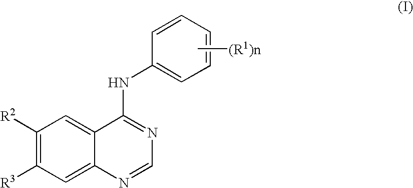
wherein n is an integer of 0 to 3, R¹ is a hydrogen atom, a halogen atom, hydroxy, cyano, nitro, trifluoromethyl, C₁₋₅ alkyl, C₁₋₅ alkoxy, —S(O)_{f}R¹³ (wherein f is an integer of 0 to 2, and R¹³ is C₁₋₅ alkyl), —NR¹⁴R¹⁵ (wherein R¹⁴ and R¹⁵ are each independently a hydrogen atom, C₁₋₅ alkyl, C₁₋₅ alkanoyl or C₁₋₅ alkylsulfonyl), C₂₋₅ alkenyl, C₂₋₅ alkynyl or C₁₋₅ alkanoyl, one of R² and R³ is
R²⁷SO₂NH— (wherein R²⁷ is C₁₋₅ alkyl optionally substituted by morpholino), (R²⁸SO₂)₂N— (wherein R²⁸ is C₁₋₅ alkyl optionally substituted by morpholino), C₁₋₅ alkoxy, CH₃COCH₂CONH—, CH₃SCH₂CH₂CONH—, NCCH₂CONH—, 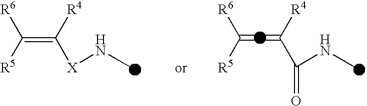
(wherein X is —C(O)— or SO₂—, and R⁴, R⁵ and R⁶ are each independently a hydrogen atom, a halogen atom or C₁₋₅ alkyl optionally substituted by a halogen atom, morpholino, 4-C₁₋₅ alkylpiperazin-1-yl or di(C₁₋₅ alkyl)amino), or 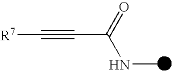
(wherein R⁷ is C₁₋₅ alkyl optionally substituted by a hydrogen atom, a halogen atom, morpholino, 4-C₁₋₅ alkylpiperazin-1-yl or di(C₁₋₅ alkyl)amino), and
the other of R² and R³ is 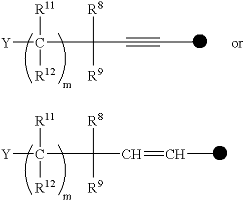
wherein R⁸ and R⁹ are each independently [a] a hydrogen atom, [b] C₁₋₅ alkyl optionally substituted by hydroxy or C₁₋₅ alkoxy, [c] R⁸ and R⁹ in combination show C═O, or [d] R⁸ and R⁹ in combination form a ring to show C₃₋₈ cycloalkylene optionally via —O—, —S— or —NR¹⁰— (wherein R¹⁰ is a hydrogen atom or C₁₋₅ alkyl), m is an integer of 0 to 3, R¹¹ and R¹² are each independently a hydrogen atom or C₁₋₅ alkyl, and
Y is a hydrogen atom, hydroxy, C₁₋₅ alkoxy, C₁₋₅ alkanoyloxy, —N(R¹⁶)—(CO)u-(CR¹⁷R¹⁸)v-(CO)j-R¹⁹ (wherein R¹⁶ is [a] a hydrogen atom or [b] C₁₋₅ alkyl optionally substituted by cyano or C₁₋₅ alkoxy, R¹⁷ and R¹⁸ are each independently a hydrogen atom or C₁₋₅ alkyl, u and j are each 0 or 1, v is an integer of 1 to 5, and R¹⁹ is a hydrogen atom, hydroxy, cyano, amino, C₁₋₅ alkoxy, morpholino, 4-C₁₋₅ alkylpiperazin-1-yl or di(C₁₋₅ alkyl)amino,
provided that when (i) u and j are simultaneously 0, then v is an integer of 2 to 5, and when (ii) R¹⁹ is a cyano group, then j is 0), 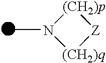
wherein p and q are each independently 2 or 3, and Z is —O—, —S(O)_{g}— (wherein g is an integer of 0 to 2), carbonyl or —NR²⁰— (wherein R²⁰ is [a] a hydrogen atom, [b] C₁₋₅ alkylsulfonyl, [c] C₁₋₅ alkanoyl, [d] C₁₋₅ alkoxycarbonyl, or [e] C₁₋₅ alkyl optionally substituted by cyano or C₁₋₅ alkoxy), or 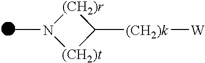
wherein r and t are each independently an integer of 1 to 3, k is 0 or 1, and W is a hydrogen atom, hydroxy, C₁₋₅ alkoxy, C₁₋₅ alkanoyloxy, carboxyl, cyano, di(C₁₋₅ alkyl)amino, morpholino, pyrrolidin-1-yl, piperidin-1-yl, 4-C₁₋₅ alkylpiperazin-1-yl or CONR²¹R²² (wherein R²¹ and R²² are each independently a hydrogen atom or C₁₋₅ alkyl), or a pharmaceutically acceptable salt thereof, or a hydrate or solvate thereof, or an optically active form or racemate thereof or a diastereomer mixture thereof.

2. The method of claim 1, wherein, in the quinazoline derivative of the aforementioned formula (I),
R² is R²⁷SO₂NH— (wherein R²⁷ is C₁₋₅ alkyl optionally substituted by morpholino), (R²⁸SO₂)₂N— (wherein R²⁸ is C₁₋₅ alkyl optionally substituted by morpholino), C₁₋₅ alkoxy, CH₃COCH₂CONH—, CH₃SCH₂CH₂CONH—, N≡CCH₂CONH—, or 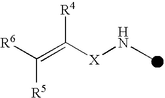
(wherein X is —C(O)— or SO₂—, and R⁴, R⁵ and R⁶ are each independently a hydrogen atom, a halogen atom or C₁₋₅ alkyl optionally substituted by a halogen atom, morpholino, 4-C₁₋₅ alkylpiperazin-1-yl or di(C₁₋₅ alkyl)amino), and
R³ is 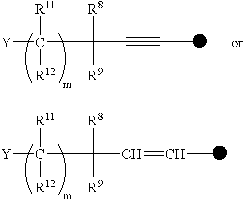
wherein m is an integer of 0 to 3,
R⁸ and R⁹ are each independently a hydrogen atom, hydroxy or C₁₋₅ alkyl optionally substituted by C₁₋₅ alkoxy, R⁸ and R⁹ in combination show —C(O)—, or R⁸ and R⁹ in combination form a ring to show C₃₋₈ cycloalkylene optionally via —O—, —S— or —NR¹⁰—
(wherein R¹⁰ is a hydrogen atom or C₁₋₅ alkyl),
R¹¹ and R¹² are each independently a hydrogen atom or C₁₋₅ alkyl, and
Y is a hydrogen atom, hydroxy, C₁₋₅ alkoxy, C₁₋₅ alkanoyloxy, —N(R¹⁶)—(CO)u-(CR¹⁷R¹⁸)v-(CO)j-R¹⁹ (wherein R¹⁶ is a hydrogen atom, or C₁₋₅ alkyl optionally substituted by cyano or C₁₋₅ alkoxy,
R¹⁷ and R¹⁸ are each independently a hydrogen atom or C₁₋₅ alkyl,
u and j are each independently 0 or 1, v is an integer of 1 to 5, and
R¹⁹ is a hydrogen atom, hydroxy, cyano, amino, C₁₋₅ alkoxy, morpholino, 4-C₁₋₅ alkylpiperazin-1-yl, or di(C₁₋₅ alkyl)amino,
provided that when (i) u and j are simultaneously 0, then v is an integer of 2 to 5, and when (ii) R¹⁹ is cyano, then j is 0), 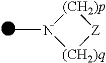
wherein p and q are each independently 2 or 3, and Z is —O—, —S(O)_{g}— (wherein g is an integer of 0 to 2), —C(O)— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or 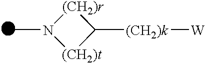
wherein r and t are each independently an integer of 1 to 3, k is 0 or 1, and W is a hydrogen atom, hydroxy, C₁₋₅ alkoxy, C₁₋₅ alkanoyloxy, carboxyl, cyano, di(C₁₋₅ alkyl)amino, morpholino, pyrrolidin-1-yl, piperidin-1-yl, 4-C₁₋₅ alkylpiperazin-1-yl or CONR²¹R²² (wherein R²¹ and R²² are each independently a hydrogen atom or C₁₋₅ alkyl).

3. The method of claim 2, wherein, in the quinazoline derivative of the aforementioned formula (I),
R³ is 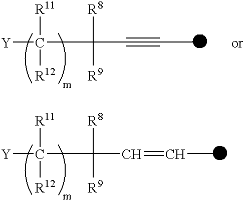
wherein m is an integer of 0 to 3,
R⁸ and R⁹ are each independently a hydrogen atom or C₁₋₅ alkyl optionally substituted by C₁₋₅ alkoxy, or R⁸ and R⁹ in combination form a ring to show C₃₋₈ cycloalkylene optionally via —O— or —NH—,
R¹¹ and R¹² are each independently a hydrogen atom or C₁₋₅ alkyl, and
Y is a hydrogen atom, hydroxy, C₁₋₅ alkoxy, C₁₋₅ alkanoyloxy, —N(R¹⁶)—(CO)u-(CR¹⁷R¹⁸)v-(CO)j-R¹⁹ (wherein R¹⁶ is a hydrogen atom, or C₁₋₅ alkyl optionally substituted by cyano or C₁₋₅ alkoxy, R¹⁷ and R¹⁸ are each independently a hydrogen atom or C₁₋₅ alkyl, u and j are each independently 0 or 1, v is an integer of 1 to 5, and R¹⁹ is a hydrogen atom, hydroxy, cyano, amino, C₁₋₅ alkoxy, morpholino, 4-C₁₋₅ alkylpiperazin-1-yl, or di(C₁₋₅ alkyl)amino, provided that when (i) u and j are simultaneously 0, then v is an integer of 2 to 5, and when (ii) R¹⁹ is cyano, then j is 0), 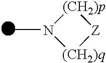
wherein p and q are each independently 2 or 3, and Z is —O—, —C(O)— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or 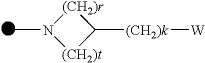
wherein r and t are each independently an integer of 1 to 3, k is 0 or 1, and W is a hydrogen atom, hydroxy, C₁₋₅ alkoxy, C₁₋₅ alkanoyloxy, carboxyl, cyano, di(C₁₋₅ alkyl)amino, morpholino or CONR²¹R²² (wherein R²¹ and R²² are each independently a hydrogen atom or C₁₋₅ alkyl).

4. The method of claim 2, wherein, in the quinazoline derivative of the aforementioned formula (I), R³ is 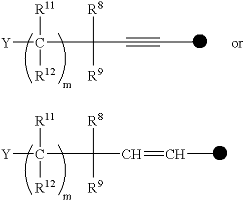
wherein m is an integer of 0 or 1,
R⁸ and R⁹ are each independently a hydrogen atom or C₁₋₅ alkyl optionally substituted by C₁₋₅ alkoxy, or R⁸ and R⁹ in combination form a ring to show C₃₋₈ cycloalkylene optionally via —O— or —NH—, and
Y is C₁₋₅ alkoxy, —N(R¹⁶)—(CO)u-(CH₂)₂—(CO)j-R¹⁹ (wherein u and j are each 0 or 1, R¹⁶ is a hydrogen atom, or C₁₋₅ alkyl optionally substituted by C₁₋₅ alkoxy, and R¹⁹ is a hydrogen atom, cyano, C₁₋₅ alkoxy, morpholino, 4-C₁₋₅ alkylpiperazin-1-yl or di(C₁₋₅ alkyl)amino, provided that when R¹⁹ is cyano, then j is 0), 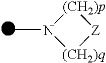
wherein p and q are each independently 2 or 3, and Z is —O— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or 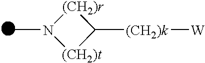
wherein r and t are each independently 1 or 2, and
W is a hydrogen atom, hydroxy, C₁₋₅ alkoxy, C₁₋₅ alkanoyloxy or carboxyl.

5. The method of claim 2, wherein, in the quinazoline derivative of the aforementioned formula (I), R³ is 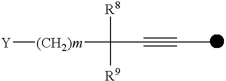
wherein m is 0 or 1,
R⁸ and R⁹ are each independently a hydrogen atom or C₁₋₅ alkyl optionally substituted by C₁₋₅ alkoxy, or R⁸ and R⁹ in combination form a ring to show C₃₋₈ cycloalkylene optionally via —O— or —NH—, and
Y is C₁₋₅ alkoxy, —N(R¹⁶)—(CH₂)₂—R¹⁹ (wherein R¹⁶ is a hydrogen atom or C₁₋₅ alkyl optionally substituted by C₁₋₅ alkoxy, and R¹⁹ is a hydrogen atom, cyano, C₁₋₅ alkoxy, morpholino, 4-C₁₋₅ alkylpiperazin-1-yl, or di(C₁₋₅ alkyl)amino, provided that when R¹⁹ is cyano, then j is 0), 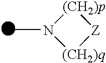
wherein p and q are each independently 2 or 3, and
Z is —O— or —NR²⁰— (wherein R²⁰ is a hydrogen atom, C₁₋₅ alkylsulfonyl, C₁₋₅ alkanoyl, C₁₋₅ alkoxycarbonyl, or C₁₋₅ alkyl optionally substituted by cyano or C₁₋₅ alkoxy), or 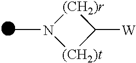
wherein r and t are each independently 1 or 2, and
W is a hydrogen atom, hydroxy, C₁₋₅ alkoxy, C₁₋₅ alkanoyloxy, carboxyl.

6. The method of claim 5, wherein, in the quinazoline derivative of the aforementioned formula (I), R² is R²⁷SO₂NH— (wherein R²⁷ is C₁₋₅ alkyl), C₁₋₅ alkoxy or 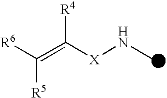
wherein each symbol is as defined above.

7. The method of claim 6, wherein, in the quinazoline derivative of the aforementioned formula (I), n is 1 or 2, and R¹ is a halogen atom, cyano, C₁₋₅ alkyl, C₁₋₅ alkoxy, —NR¹⁴R¹⁵ (wherein R¹⁴ and R¹⁵ are each independently a hydrogen atom, C₁₋₅ alkyl, C₁₋₅ alkanoyl or C₁₋₅ alkylsulfonyl), C₂₋₅ alkynyl, or C₁₋₅ alkanoyl.

8. The method of claim 7, wherein, in the quinazoline derivative of the aforementioned formula (I), n is 2, R¹ is a halogen atom, R² is 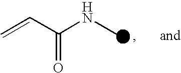
R³ is 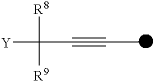
wherein R⁸ and R⁹ are each C₁₋₅ alkyl optionally substituted by C₁₋₅ alkoxy, and
Y is —N(R¹⁶)—(CH₂)₂—R¹⁹ (wherein R¹⁶ is C₁₋₅ alkyl optionally substituted by C₁₋₅ alkoxy, and R¹⁹ is di(C₁₋₅ alkyl)amino), or 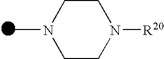
wherein R²⁰ is C₁₋₅ alkyl optionally substituted by C₁₋₅ alkoxy.

9. The method of claim 8, wherein the quinazoline derivative is N-{4-[(3-chloro-4-fluorophenyl)amino]-7-[3-methyl-3-(4-methyl-1-piperazinyl)-1-butynyl]-6-quinazolinyl}acrylamide bis(4-methylbenzenesulfonate).

10. (canceled)

11. The method of claim 1, wherein the disease involving a heat shock protein is a hormone-dependent disease, a hematopoietic tumor or a malignant soft tissue tumor.

12. The method of claim 11, wherein the hormone-dependent disease is prostatic hyperplasia, endometriosis or a hormone-sensitive cancer.

13. The method of claim 12, wherein the hormone-sensitive cancer is a hormone-sensitive breast cancer, a hormone-sensitive prostate cancer or a hormone-sensitive endometrial cancer.

14. The method of claim 11, wherein the hematopoietic tumor is leukemia, malignant lymphoma or myeloma.

15. The method of claim 11, wherein the malignant soft tissue tumor is osteosarcoma or soft tissue sarcoma.

16-21. (canceled)
